(19) United States
(12) Patent Application Publication
(10) Pub. No.: US 2020/0062839 A1
(43) Pub. Date: Feb. 27, 2020

(54) USE OF DIANHYDROGALACTITOL OR ANALOGS AND DERIVATIVES IN COMBINATION WITH VEGF INHIBITORS TO TREAT CANCER

(71) Applicant: DEL MAR PHARMACEUTICALS (BC) LTD., Vancouver (CA)

(72) Inventors: Jeffrey A. BACHA, Vancouver (CA); Dennis M. BROWN, Menlo Park, CA (US); Anne STEINØ, Vancouver (CA)

(21) Appl. No.: 16/609,721

(22) PCT Filed: May 1, 2018
(86) PCT No.: PCT/US2018/030391

§ 371 Date: Oct. 30, 2019

Related U.S. Application Data
(60) Provisional application No. 62/492,647, filed on May 1, 2017.
(60) Provisional application No. 62/660,029, filed on Apr. 19, 2018.

(51) Int. Cl. C07K 16/22; A61K 31/336; A61K 31/047; A61P 35/00

(52) U.S. Cl. CPC C07K 16/22; A61P 35/00; A61K 31/047; A61K 31/336

(57) ABSTRACT

Methods and compositions employing dianhydrogalactitol or a derivative or analog of dianhydrogalactitol together with a vascular endothelial growth factor (VEGF) inhibitor or other anti-neoplastic agents can be used for treatment of glioma. In particular, methods and compositions according to the invention can decrease the invasiveness of gliomas. Further disclosed are methods of using the compositions for treating a malignancy that has failed a VEGF inhibitor treatment.

### CROSS-REFERENCES TO RELATED APPLICATIONS

This application claims the benefit of U.S. Provisional Application No. 62/492,647 filed May 1, 2017 and U.S. Provisional Application No. 62/660,029 filed Apr. 19, 2018, the disclosures of which are expressly incorporated herein by reference in their entirety.

### BACKGROUND OF THE INVENTION

VEGF inhibitors, such as bevacizumab, are frequently used as a therapeutic agent to treat glioma. Although such VEGF inhibitors can be effective antineoplastic agents for gliomas and often provide initial tumor response and disease control, the effects are transient and tumors recur after a median of 3-5 months. In addition, treatment with VEGF inhibitors lead to increased metastatic properties and invasiveness of gliomas (see e.g., J. Ebos et al., “Accelerated Metastasis After Short-Term Treatment with a Potent Inhibitor of Tumor Angiogenesis,” Cancer Cell 15: 232-239 (2009); M. Pàes-Ribes et al., “Antiangiogenic Therapy Elicits Malignant Progression of Tumors to Increased Local Invasion and Distant Metastasis,” Cancer Cell 15: 220-231 (2009); O. Keunen et al., “Anti-VEGF Treatment Reduces Blood Supply and Increases Tumor Cell Invasion in Glioblastoma,” Proc. Natl. Acad. Sci. USA 108: 3749-3754 (2011))).

It is known that VEGF inhibitors create normalization of the blood vessels (reducing the disorganized hypervascularization that occurs in glioblastoma tumors), which causes the cancer cells to enter a hypoxic state (S. P. Niclou et al., “Anti-VEGF Treatment Reduces Blood Supply and Increases Tumor Cell Invasion in Glioblastoma,” Proc. Natl. Acad. Sci. USA 108: 3749-54 (2011)). This intensifies a metabolic shift of the cancer cells from oxidative phosphorylation to anaerobic glycolysis and lactic acid fermentation (the Warburg effect). While initially limiting the energy supply to the cancer cells, continued hypoxia causes metabolic adaption in the cancer cells and has been shown to lead to increased invasiveness of the glioma (F. Fack et al., “Bevacizumab Treatment Induces Metabolic Adaptation Toward Anaerobic Metabolism in Glioblastoma,” Acta Neuropathol. 129: 115-131 (2015); S. P. Niclou et al., “Anti-VEGF Treatment Reduces Blood Supply and Increases Tumor Cell Invasion in Glioblastoma,” Proc. Natl. Acad. Sci. USA 108: 3749-54 (2011)).

Glioblastoma is the most common and aggressive malignant primary brain tumor occurring in humans. Glioblastoma involves glial cells; it accounts for 52% of all functional tissue brain tumor cases and 20% of all intracranial tumors. Its estimated frequency of occurrence is 2-3 cases per 100,000 people in Europe and North America.

Glioblastoma has an extremely poor prognosis, despite various treatment methods including open craniotomy with surgical resection of as much of the tumor as possible, followed by sequential or concurrent chemoradiotherapy with temozolomide, antiangiogenic therapy with bevacizumab, gamma knife radiosurgery, and symptomatic management with corticosteroids. The median survival time for glioblastoma patients is only 14 months from initial diagnosis.

Cancer cells with stem-cell-like properties have been found in glioblastomas. These cells are believed to give rise to the brain tumor, have a higher level of resistance to standard of care chemotherapies, increased tumorigenic potential and increased metabolic adaptation abilities (W. A. Flavahan et al., “Brain Tumor Initiating Cells Adapt to Restricted Nutrition Through Preferential Glucose Uptake,” Nat. Neurosci. 16(10):1373-1382 (2013); Y. P. Ramirez et al., “Glioblastoma Multiforme Therapy and Mechanisms of Resistance,” Pharmaceuticals 6(12):1475-1506 (2013)). Cancer stem cells (CSCs) may be particularly adapted to grow under hypoxic conditions as created by VEGF inhibitor treatment (R. Bjerkvig et al., “Cancer Stem Cells and Angiogenesis,” Semin. Cancer Biol. 19: 279-284 (2009)). This may partly be due to rapid CSC adaptation to hypoxic environment by increasing glucose uptake to support high rates of glycolysis. In hypoxic and acidic microenvironments, glioblastoma CSCs have been shown to increase their expression of intratumoral glucose receptors GLUT1 and GLUT3 by 20% and 300%, respectively, and increase their tumorigenic potential relative to glioblastoma CSCs under normoxic (normal oxygen level) conditions (W. A. Flavahan et al., “Brain Tumor Initiating Cells Adapt to Restricted Nutrition Through Preferential Glucose Uptake,” Nat. Neurosci. 16(10):1373-1382 (2013)). This may be one cause of the resistance of glioblastoma to conventional chemotherapeutic treatment regimens and its high recurrence rate.

A recently approved therapeutic approach to glioblastoma involves the use of the monoclonal antibody bevacizumab, which is a humanized monoclonal antibody that inhibits vascular endothelial growth factor A (VEGF-A) and thus acts as an angiogenesis inhibitor. Although bevacizumab may retard the initial progression of the disease, neither first-line use of bevacizumab in patients with newly diagnosed glioblastoma nor second-line use in recurrent glioblastoma improved overall survival (M. R. Gilbert et al., “A Randomized Trial of Bevacizumab for Newly Diagnosed Glioblastoma,” New Engl. J. Med. 370: 699-708 (2014); W. Taal et al., “Single-Agent Bevacizumab or Lomustine Versus a Combination of Bevacizumab Plus Lomustine in Patients with Recurrent Glioblastoma (BELOB Trial): A Randomised Controlled Phase 2 Trial,” The Lancet 15(9): 943-53 (2014)). This might be due to the rapid adaptation of glioblastoma CSCs to the bevacizumab-induced hypoxic conditions. Additionally, unlike some other malignancies in which the use of bevacizumab results in a potentiation of chemotherapy, in glioblastoma, the addition of chemotherapy to bevacizumab did not improve on results from bevacizumab alone. Bevacizumab reduces brain edema and consequent symptoms, and it may be that the benefit on quality of life from this drug is due to its action against edema rather than any action against the tumor itself. Patients in which both temozolomide and bevacizumab have been ineffective have few if any treatment options.

There is, therefore, a need for improved treatment for gliomas that can suppress invasiveness of the gliomas and be used in conjunction with conventional treatments for these malignancies.

### SUMMARY OF THE INVENTION

Disclosed herein are methods and compositions employing a DNA crosslinking hexitol derivative, such as dianhydrogalactitol or a derivative or analog of dianhydrogalactitol, together with a VEGF inhibitor for treatment of a malignancy. Typically, the malignancy is a central nervous system malignancy; the central nervous system malignancy can be glioma, glioblastoma, or medulloblastoma. In another alternative, the DNA crosslinking hexitol derivative can be employed together with another anti-neoplastic agent. In particular, methods and compositions according to the invention can decrease the invasiveness of gliomas and other types of malignancies.

One aspect of the present invention is a method for treatment of a malignancy comprising administration of:

(1) a therapeutically effective quantity of a hexitol derivative selected from the group consisting of dianhydrogalactitol, a derivative or analog of dianhydrogalactitol, diacetyldianhydrogalactitol, a derivative or analog of diacetyldianhydrogalactitol, dibromodulcitol, and a derivative or analog of dibromodulcitol; and

(2) a therapeutically effective quantity of a VEGF inhibitor.

In some embodiments, the administration of the therapeutically effective quantities of the hexitol derivative and the VEGF inhibitor treats the malignancy by reducing the invasiveness of the malignancy. As described above, typically, the malignancy is a central nervous system malignancy. The central nervous system malignancy can be glioma, glioblastoma, or medulloblastoma.

Typically, the DNA crosslinking hexitol derivative is selected from the group consisting of dianhydrogalactitol and a derivative or analog of dianhydrogalactitol. Preferably, the hexitol derivative is dianhydrogalactitol.

In one alternative, the hexitol derivative acts together with the administration of a VEGF inhibitor and the induction of hypoxia, through upregulation of the glucose transporters including GLUT1 or GLUT3 on GBM tumor cells. In this alternative, typically, the increase in the uptake of glucose and other compounds that bind to the glucose transporters, including the hexitol derivative, increases the quantity of the hexitol derivative getting into the tumor cells and thus the cell kill activity, without increasing the administered dose and systemic toxicity of the hexitol derivative. In still another alternative, the hexitol derivative is administered concurrently with or immediately following administration of a VEGF inhibitor to increase the uptake of the dianhydrogalactitol or derivative or analog thereof in glioblastoma CSCs thereby increasing cell kill activity against CSCs, thereby reducing CSC-related tumor recurrence and improving outcome of the disease. In yet another alternative, the administration of dianhydrogalactitol increases the extent of double-strand DNA breaks when cells, to which the dianhydrogalactitol and the VEGF inhibitor have been administered, resume DNA synthesis; the cells can be CSCs.

The VEGF inhibitor can, in one alternative, be a PPAR activator or agonist that indirectly inhibits the activity of VEGF.

The method can further comprise the administration of a therapeutically effective quantity of an additional agent. The additional agent can be selected from the group consisting of: (i) an inhibitor of hexokinase II; (ii) a PFKFB3 inhibitor; (iii) an inhibitor of GAPDH; (iv) an inhibitor of PK-M2; (v) an inhibitor of glucose-6-phosphate isomerase; (vi) an inhibitor of LDH; (vii) an inhibitor of aldolase; (viii) an inhibitor of phosphoglycerate mutase; (ix) an inhibitor of enolase; (x) a MCT inhibitor; (xi) an agent that inhibits conversion of glutamate to α-ketoglutarate; (xii) an inhibitor of the cysteine-glutamate antiporter; (xiii) an inhibitor of NamPRT; (xiv) an agent to either restore p53 function or suppress activity of mutated p53; and (xv) an inhibitor of the pentose phosphate pathway (PPP).

In another alternative, the method further comprises the administration of a therapeutically effective quantity of an additional agent having anti-neoplastic activity against glioma.

Another aspect of the invention is a method for treatment of a malignancy comprising administration of:

(a) a therapeutically effective quantity of a hexitol derivative selected from the group consisting of dianhydrogalactitol, a derivative or analog of dianhydrogalactitol, diacetyldianhydrogalactitol, a derivative or analog of diacetyldianhydrogalactitol, dibromodulcitol, and a derivative or analog of dibromodulcitol; and

a therapeutically effective quantity of an additional agent, wherein the additional agent is selected from the group consisting of:

(i) a biguanide;

(ii) a HKII inhibitor;

(iii) a PFKFB3 inhibitor;

(iv) a GAPDH inhibitor;

(v) a glucose-6-phosphate isomerase inhibitor;

(vi) a LDH inhibitor;

(vii) an aldolase inhibitor;

(viii) a phosphoglycerate mutase inhibitor;

(ix) an enolase inhibitor;

(x) a pyruvate dehydrogenase kinase inhibitor;

(xi) a pentose phosphate pathway inhibitor;

(xii) a MCT inhibitor;

(xiii) an agent that inhibits conversion of glutamate to α-ketoglutarate;

(xiv) an agent that inhibits the cysteine-glutamate antiporter;

(xv) a PPAR activator;

(xvi) a NamPRT inhibitor;

(xvii) an agent to either restore p53 function or suppress activity of mutated p53; and

(xviii) an inhibitor of the pentose phosphate pathway. As stated above, typically, the malignancy is a central nervous system malignancy; the central nervous system malignancy can be glioma, glioblastoma, or medulloblastoma.

### DESCRIPTION OF THE DRAWINGS

The foregoing aspects and many of the attendant advantages of this invention will become more readily appreciated as the same become better understood by reference to the following detailed description, when taken in conjunction with the accompanying drawings, wherein:

FIG. 1A is a series of graphs showing the effect of dianhydrogalactitol on DNA repair, DNA damage, and cell cycling in hypoxic and normoxic cells of the U251 human glioblastoma cell line, with the data obtained by flow cytometry. ATMP denotes phosphorylated (=activated) ATM (ataxia-telangiectasia mutated) which is a kinase that is part of the homologous recombination (HR) DNA damage repair pathway responsible for repair of DNA double strand breaks; its presence is a marker for DNA repair, specifically through the HR pathway. PE is a fluorochrome that is used to detect the ATMP in flow cytometry. H2AXP is activated H2AX which is a histone marker for DNA double strand breaks (DNA damage). AF647 is a fluorochrome used for detection of activated H2AX in flow cytometry. DAPI is a fluorescent stain that binds to DNA and thus is a marker for the amount of DNA that therefore can be used to detect cell cycle changes as the amount of DNA doubles through the S-phase and into the G2/M phase. The leftmost panel shows the extent of DNA repair, the center panel shows the extent of DNA damage, specifically double stand breaks, and the rightmost panel shows the proportion of cells at each point in the cell cycle. The top panel shows the isotype antibody control. The following legends designating treatment of the cells are interpreted as follows: (i) “Normoxia CTR” is normoxic cells as a control without no dianhydrogalactitol treatment; (ii) “Hypoxia CTR” is hypoxic cells as a control without no dianhydrogalactitol treatment; (iii) “Normoxia 1 h” is normoxic cells after 1 hour of treatment with dianhydrogalactitol; (iv) “Hypoxia 1 h” is hypoxic cells after 1 hour of treatment with dianhydrogalactitol; (v) “Normoxia 1 h+24 h” is normoxic cells after a 1-hour pulse with dianhydrogalactitol followed by a 24-hour washout; (vi) “Hypoxia 1 h+24 h” is hypoxic cells after a 1-hour pulse with dianhydrogalactitol followed by a 24-hour washout; (vii) “Normoxia 1 h+48 h” is normoxic cells after a 1-hour pulse with dianhydrogalactitol followed by a 48-hour washout; and (vii) “Hypoxia 1 h+48 h” is hypoxic cells after a 1-hour pulse with dianhydrogalactitol followed by a 48-hour washout.

FIGS. 1B-1D is a series of graphs showing the effect of dianhydrogalactitol on DNA repair, DNA damage, and cell cycling in hypoxic and normoxic cells of the U251 human glioblastoma cell line. For each pair of bars, the left member of the pair (white bar) is for normoxic cells, while the right member of the pair (striped bar) is for hypoxic cells. In FIG. 1B the fraction of cells with high ATMP is shown. In FIG. 1C the fraction of cells with high H2AXP is shown, and in FIG. 1D the fraction of cells in the S/G2/M phases of the cell cycle (cells that are replicating or have replicated DNA) is shown.

FIG. 2A demonstrates cytotoxic effects of increasing concentrations (1-1000 μM) dianhydrogalactitol on primary GBM organotypic spheroids of MGMT-unmethylated T16 (top panels) or MGMT-methylated P3 (bottom panels) origin, in vitro. Representative images of treated spheroids are presented.

FIG. 2B is a graph showing T16 GBM cell viability and cell death displayed as percentage of viable and dead cells, respectively, versus dianhydrogalactitol concentration. Percentages were normalized to the control untreated spheroids. The calculated IC₅₀ value for dianhydrogalactitol is shown on the graph.

FIG. 2C is a graph showing P3 GBM cell viability and cell death displayed as percentage of viable and dead cells, respectively, versus dianhydrogalactitol concentration. Percentages were normalized to the control untreated spheroids. The calculated IC₅₀ for dianhydrogalactitol is shown on the graph.

FIG. 3 depicts the study design and timeline of the in vivo experimental set-up and treatment groups.

FIG. 4 shows examples of intracranial T16 GBM tumor volumes in nude mice as evaluated by MRI T2 at day 35. Small tumors were detected in the majority of mice and mice were randomized to 4 treatment groups accordingly.

FIG. 5 shows examples of intracranial T16 GBM tumor volumes in control and treatment group as evaluated by MRI T2 at day 56 (end of study). Smaller tumors were detected by T2 MRI in dianhydrogalactitol treated groups compared to Control and bevacizumab-treated mice. T1+contrast (T1+C) MRI showed decreased contrast in bevacizumab treatment groups compared to control mice and dianhydrogalactitol treatment group.

FIG. 6A shows comparison of T16 GBM tumor volumes as detected by MRI in nude mice between the 4 treatment groups on Days 49 and 56 of the experiment. Dianhydrogalactitol treatment groups had significantly smaller tumors at both time points compared to the Control and bevacizumab group. No significant difference was observed between mice treated with dianhydrogalactitol alone and dianhydrogalactitol in combination with bevacizumab at the two time points (Day 49 and 56). This was likely due to the high efficacy of dianhydrogalactitol treatment alone. However importantly, a tendency towards smaller tumors was observed upon combined treatment at both time points.

FIG. 6B is a table of mean tumor volumes between the four experimental groups on Days 35, 49, and 56 of the experiment.

FIG. 6C shows tumor progression over time in the four experimental groups. All groups had similar tumor volume at the start of treatment (MRI2, day 35). Tumor progression was measured in two consecutive MRIs (MRI3 day 49, MRI4 day 56) and mean tumor volumes per group (mm³) were plotted against time. Dianhydrogalactitol treatment groups had slower tumor growth compared to the Control and Bevacizumab group.

FIG. 6D compares tumor growth rate in percent growth per day between dianhydrogalactitol treatment groups. Tumor progression was calculated for entire study (day 35 vs. day 56) and for last 7 days of treatment (day 49 vs. day 56) with the formula: 100*log (TVf/TV0)/(tf−t0), where TVf is tumor volume at end time point, TV0 is tumor volume at start time point, tf is end time point, t0 is start time point. The data shows significantly reduced tumor progression over time for dianhydrogalactitol combined treatment with Bevacizumab (squared, right) compared to dianhydrogalactitol treatment alone (circles, left) for both time periods (entire study and last 7 days).

### DETAILED DESCRIPTION OF THE INVENTION

Disclosed herein are methods and compositions employing a DNA crosslinking hexitol such as dianhydrogalactitol or a derivative or analog of dianhydrogalactitol together with a VEGF inhibitor or other anti-neoplastic agents can be used for treatment of a malignancy that substantially employs glycolysis for energy generation. Typically, the malignancy is a central nervous system malignancy; the central nervous system malignancy can be glioma, glioblastoma, or medulloblastoma. In particular, methods and compositions according to the invention can decrease the invasiveness of gliomas.

Glucose metabolism in cancer cells is primarily characterized by two major biochemical events: (i) increased glucose uptake and (ii) increased aerobic glycolysis, the process of conversion of glucose into pyruvate eventually resulting in the production of lactate (fermentation) and an acidic microenvironment. It has long been known that cancer cells are distinguished from non-malignant cells by their increased reliance on glycolysis for energy production and decreased reliance on oxidative phosphorylation. The existence of a link between aerobic glycolysis and tumorigenesis has long been known as the “Warburg effect,” following studies by the Nobel-prize-winning German biochemist. However, the underlying mechanistic details pertinent to the causes and consequences of this metabolic phenotype in cancer cells remained unclear. Conceptual advances in the past decades have improved the understanding on the biological significance of tumor metabolism, including the increased reliance of cancer cells on glycolysis for energy production. As a result, deregulated or altered energy metabolism has been recognized as one of the hallmarks of cancer.

It is therefore increasingly evident that the cancer-specific upregulation of glycolysis is regulated through oncogenes (e.g. c-myc or Akt; Akt can act as an oncogene in certain circumstances). The effect of the tumor microenvironment has been experimentally verified using three-dimensional in vitro models, where spheroid formation resulted in the generation of a central hypoxic area eventually leading to an increase in glycolytic flux. Akt, the serine/threonine kinase, is an oncogene that promotes cancer growth. Akt activates aerobic glycolysis, and importantly, renders cancer cells dependent on glycolysis for survival. There are additional regulatory pathways involved. Coordinated networks involving signaling pathways enable cancer cells to detect and integrate their immediate environmental conditions to balance their anabolic and catabolic processes. The mammalian Target of Rapamycin (mTOR) represents such a pathway where the intracellular energy sensing molecule AMPK can impact the mTOR complex I (mTORC1) mechanism of activation to either delay or halt energy-consuming synthetic processes. Such an adaptation involves mTORC1-mediated regulation of the expression of glycolytic enzymes through the activation of genes such as c-myc and HIF1-α. It has also been realized that both oncogenes and tumor suppressors regulate altered energy metabolism. Oncogenic mutations can cause the upregulation of glucose transporter genes such as GLUT1 or GLUT3, increased glucose consumption by cancer cells, which in turn increases the rate of glucose metabolism by such cells. Conversely, the glycolytic/metabolic phenotype confers selective advantage to cancer cells by supporting uninterrupted growth of the cells. For example, a higher glycolytic rate in tumor cells has been shown to promote resistance to chemotherapeutic agents. In the cervical cancer cell line HeLa, for example, the enzyme pyruvate dehydrogenase kinase (PDK) isoforms PDK1 and PDK3 have been demonstrated to provide resistance to chemotherapeutic agents. Similarly, in the colon carcinoma cell line LoVo it has been demonstrated that increased aerobic lactate production (glycolysis) correlated with drug resistance. Thus, the interruption or disruption of glucose uptake and increased glycolysis in tumor cells can have two effects: (1) it can impact tumor growth by energy depletion and (2) it can sensitize or re-sensitize the tumor cells to the action of antineoplastic agents.

Methods and compositions according to the present invention can act by one or more of the following mechanisms: (1) administration of bevacizumab or another VEGF inhibitor induces hypoxia, upregulation of the glucose transporters, such as, but not limited to, GLUT1 and GLUT3 on glioma cancer cells and CSCs, which in turn causes an increase in the uptake of glucose and of other compounds that bind to the glucose transporters, including dianhydrogalactitol and derivatives thereof; (2) blockage of uptake of glucose to deprive the malignant cells of an energy source; or (3) increased uptake of dianhydrogalactitol or derivatives thereof due to increased metabolism in the tumor, especially in an instance where the tumor changes from a proliferative phenotype to an invasive phenotype. Although the inventors believe that one or more of these mechanisms can contribute to the anti-neoplastic activity of compositions or methods according to the present invention, the inventors are not bound thereby, and other mechanisms may be involved.

VEGF is typically upregulated in gliomas (H. Miletic et al., “Anti-VEGF Therapies for Malignant Glioma: Treatment Effects and Escape Mechanisms,” Expert Opin. Ther. Targets 13: 455-468 (2007)). VEGF is transcriptionally upregulated under hypoxic conditions by hypoxia-inducible factor 1α (HIF-1α). HIF-1α is stabilized under hypoxic conditions and dimerizes with HIF-2α to activate the VEGF-A promoter. Additionally, overexpression of oncogenes such as Src or ras or the absence of activity of tumor suppressor genes such as p53 can also upregulate VEGF transcription. Additionally, several growth factors, including EGF, PDGF, fibroblast growth factor 2 (FGF2), TGF-β, and various cytokines can also stimulate VEGF expression. VEGF also promotes expression of anti-apoptotic factors such as bcl2, survivin, and A1.

VEGF signals through its major receptors VEGFR-1 and VEGFR-2. The induction of angiogenesis is mainly mediated through VEGFR-2, which activates Akt and MEK-regulated extracellular-regulated MAP kinase (Erk) pathways.

Some VEGF inhibitors that can be used in the methods and compositions disclosed herein include: (1) bevacizumab (Avastin) (monoclonal antibody); (2) ranibizumab (Lucentis) (monoclonal antibody Fab fragment); (3) lapatinib (tyrosine kinase inhibitor); (4) sunitinib (tyrosine kinase inhibitor) (5) sorafenib (tyrosine kinase inhibitor) (6) axitinib (tyrosine kinase inhibitor); (7) pazopanib (tyrosine kinase inhibitor); (8) tetrahydrocannabinol; (9) cannabidiol; (10) thiazolidinediones (including the following agents: rosiglitazone, pioglitazone, lobeglitazone, troglitazone, netoglitazone, rivoglitazone, and ciglitazone); and (11) withaferin A. Other VEGF inhibitors are also known in the art.

The molecular structure of dianhydrogalactitol—and derivatives thereof—is a substituted hexitol which resembles the hexose structure of the glucose molecule. Due to the similar molecular structure, dianhydrogalactitol and derivatives thereof may be able to use glucose transporters to enter the cancer cells. Although the inventors are not bound by this hypothesis, if dianhydrogalactitol or a derivative or analog thereof is administered in combination with or immediately following bevacizumab or another VEGF inhibitor, the uptake of dianhydrogalactitol or the derivative or analog thereof can be increased due to the upregulation of one or more glucose transporters. This increased uptake would in turn increase the amount of drug getting into the tumor cells and thus the cell kill activity, without increasing the administered dose and systemic toxicity of dianhydrogalactitol. Additionally, dianhydrogalactitol has shown cell kill activity in glioblastoma cancer stem cells (CSCs) with IC₅₀ values within the expected concentration range in the CNS. CSC cells are extremely chemoresistant and are known to upregulate glucose transporters in response to hypoxia. So, there is a rationale for administration of dianhydrogalactitol or a derivative or analog thereof concurrent with or immediately following bevacizumab or another VEGF inhibitor, as this would increase the uptake of the dianhydrogalactitol or derivative or analog thereof in glioblastoma CSCs thereby increasing cell kill activity against CSCs, slowing tumor growth and improving outcome of the disease. The results from the in vivo study reported herein support this hypothesis.

As the thiazolidinediones also act as PPAR activators, PPAR activators can indirectly inhibit VEGF.

The kinase inhibitors act indirectly on the activity of VEGF receptors rather than VEGF itself, as VEGF does not itself possess kinase activity. However, signal transmission by VEGF receptors depends on their kinase activity, so that inhibitors of such kinase activity block such signal transmission.

When cells go into a hypoxic state, they produce HIF-1α (hypoxia-inducible factor-la), a transcription factor that stimulates the release of VEGF-A.

Additionally, the tumor suppressor gene TP53 is mutated in a substantial fraction of glioblastomas, predominantly in the proneural subtype. Mutation in the TP53 gene leads to malignancy and tumorigenicity, primarily because of the loss of the transcription function of wild-type TP53. Additionally, mutant TP53 may also gain new functions conferring increased cell survival and cell growth, decreased apoptosis, and drug resistance. This may also lead to increased MGMT expression, which acts to repair DNA damage induced by anti-neoplastic agents such as temozolomide (X. Wang et al., “Gain of Function of Mutant TP53 in Glioblastoma: Prognosis and Response to Temozolomide,” Ann. Surg. Oncol. 21: 1337-1344 (2014)). TP53 has emerged as a therapeutic target, as described in B. Hong et al., “Targeting Tumor Suppressor p53 for Cancer Therapy: Strategies, Challenges and Opportunities,” Curr. Drug. Targets 15: 80-89 (2014). A number of approaches exists for therapeutic modalities associated with p53, including: (1) gene therapy to restore p53 function; (2) inhibition of p53-Mdm2 interaction by agents including nutlin 3, RITA (5,5′-(2,5-furandiyl)bis-2-thiophenemethanol), MI-219 (a spiro-oxindole), RG7112 (((4S,5R)-2-(4-tert-butyl-2-ethoxyphenyl)-4,5-bis(4-chlorophenyl)-4,5-dimethyl-4,5-dihydroimidazol-1-yl)(4-(3-(methyl sulfonyl)propyl)piperazin-1-yl)methanone) benzodiazepinone, NSC279287 (N-[4-[2-(3,5-dioxo-1,2-diphenyl-pyrazolidin-4-ylidene)hydrazinyl]phenyl]sulfonyl-4-propan-2-yloxy-benzamide), and NSC66811 (2-methyl-7-[phenyl(phenylamino)methyl]-8-quinolinol); (3) restoration of wild-type p53 activity in mutant p53 molecules by agents including PRIMA-1, PhiKan083, NSC319726 (1-azetidinecarbothioic acid [1-(2-pyridinyl)ethylidene]hydrazide), CP31398 (N′-[2-[2-(4-methoxyphenyl)ethenyl]-4-quinazolinyl]-N,N-dimethyl-1,3-propanediamine dihydrochloride), SCH529074 (N3-[2-[[4-[Bis(4-chlorophenyl)methyl]-1-piperazinyl]methyl]-4-quinazolinyl]-N1,N1-dimethyl-1,3-propanediamine), ellipticine, WR1065 (2[(3-aminopropyl)amino]ethanethiol dihydrochloride), and p53R3; (4) targeting p53-family proteins by agents including RETRA (2-[(4,5-dihydro-2-thiazolyl)thio]-1-(3,4-dihydroxyphenyl)ethanone hydrochloride) and 37AA (a p53-derived cyclic apoptotic peptide); (5) eliminating mutant p53 by agents including tanespimycin and 37AA; and (6) use of p53 mimetics.

Accordingly, the following combinations can be used to treat malignancies susceptible to disruption of glycolysis, in particular, central nervous system malignancies, including gliomas. These combinations include one or more DNA crosslinking hexitol derivatives as described in further detail below: (1) a DNA crosslinking hexitol derivative and a VEGF inhibitor; (2) a DNA crosslinking hexitol derivative and a biguanide; (3) a DNA crosslinking hexitol derivative, a VEGF inhibitor, and a biguanide; (4) a DNA crosslinking hexitol derivative and a HKII inhibitor; (5) a DNA crosslinking hexitol derivative, a VEGF inhibitor, and a HKII inhibitor; (6) a DNA crosslinking hexitol derivative and a PFKFB3 inhibitor; (7) a DNA crosslinking hexitol derivative, a VEGF inhibitor, and a PFKFB3 inhibitor; (8) a DNA crosslinking hexitol derivative and a GAPDH inhibitor; (9) a DNA crosslinking hexitol derivative, a VEGF inhibitor, and a GAPDH inhibitor; (10) a DNA crosslinking hexitol derivative and a glucose-6-phosphate isomerase inhibitor; (11) a DNA crosslinking hexitol derivative, a VEGF inhibitor, and a glucose-6-phosphate isomerase inhibitor; (12) a DNA crosslinking hexitol derivative and a LDH inhibitor; (13) a DNA crosslinking hexitol derivative, a VEGF inhibitor, and a LDH inhibitor; (14) a DNA crosslinking hexitol derivative and an aldolase inhibitor; (15) a DNA crosslinking hexitol derivative, a VEGF inhibitor, and an aldolase inhibitor; (16) a DNA crosslinking hexitol derivative and a phosphoglycerate mutase inhibitor; (17) a DNA crosslinking hexitol derivative, a VEGF inhibitor, and a phosphoglycerate mutase inhibitor; (18) a DNA crosslinking hexitol derivative and an enolase inhibitor; (19) a DNA crosslinking hexitol derivative, a VEGF inhibitor, and an enolase inhibitor; (20) a DNA crosslinking hexitol derivative and a pyruvate dehydrogenase kinase inhibitor; (21) a DNA crosslinking hexitol derivative, a VEGF inhibitor, and a pyruvate dehydrogenase kinase inhibitor; (22) a DNA crosslinking hexitol derivative and a pentose phosphate pathway inhibitor; (23) a DNA crosslinking hexitol derivative, a VEGF inhibitor, and a pentose phosphate pathway inhibitor; (24) a DNA crosslinking hexitol derivative and a MCT inhibitor; (25) a DNA crosslinking hexitol derivative, a VEGF inhibitor, and an MCT inhibitor; (26) a DNA crosslinking hexitol derivative and an agent that inhibits conversion of glutamate to α-ketoglutarate; (27) a DNA crosslinking hexitol derivative, a VEGF inhibitor, and an agent that inhibits conversion of glutamate to α-ketoglutarate; (28) a DNA crosslinking hexitol derivative and an agent that inhibits the cysteine-glutamate antiporter; (29) a DNA crosslinking hexitol derivative, a VEGF inhibitor, and an agent that inhibits the cysteine-glutamate antiporter; (30) a DNA crosslinking hexitol derivative and a PPAR activator; (31) a DNA crosslinking hexitol derivative, a VEGF inhibitor, and a PPAR activator; (32) a DNA crosslinking hexitol derivative and a NamPRT inhibitor; (33) a DNA crosslinking hexitol derivative, a VEGF inhibitor, and a NamPRT inhibitor; (34) a DNA crosslinking hexitol derivative and an agent to either restore p53 function or suppress activity of mutated p53; and (35) a DNA crosslinking hexitol derivative, a VEGF inhibitor, and an agent to either restore p53 function or suppress activity of mutated p53.

When multiple therapeutic agents are administered, each therapeutic agent can be administered separately, or two or more therapeutic agents can be administered in a single pharmaceutical composition. For example, when three therapeutic agents are to be administered, the following possibilities exist. (1) Each of the three therapeutic agents is administered individually; in this case, each agent can be administered in a separate pharmaceutical composition or as the agent alone without use of a pharmaceutical composition for the agent. Further details on the composition and preparation of pharmaceutical compositions are provided below. In this alternative, zero, one, two, or three separate pharmaceutical compositions can be used. (2) Two of the therapeutic agents are administered together in a single pharmaceutical composition, while the third therapeutic agent is administered separately, either as the agent alone or in a separate pharmaceutical composition. (3) All three therapeutic agents are administered together in a single pharmaceutical composition.

### DNA Crosslinking Hexitols

For the aspects and embodiments described herein related to compositions and methods of use of an DNA crosslinking hexitol, typically, the DNA crosslinking hexitol is selected from the group consisting of dianhydrogalactitol, derivatives of dianhydrogalactitol, di acetyl dianhydrogalactitol, derivatives of diacetyldianhydrogalactitol, dibromodulcitol, and derivatives of dibromodulcitol, unless otherwise specified. Preferably, the DNA crosslinking hexitol is dianhydrogalactitol, unless otherwise specified. In some instances, derivatives of dianhydrogalactitol such as compound analogs or prodrugs are preferred, as stated below.

DNA crosslinking hexitols that can be used in compositions and methods according to the present invention include galactitols, substituted galactitols, dulcitols, and substituted dulcitols. Typically, the DNA crosslinking hexitol is selected from the group consisting of dianhydrogalactitol, derivatives of dianhydrogalactitol, analogs of dianhydrogalactitol, di acetyl dianhydrogalactitol, derivatives of diacetyldianhydrogalactitol, analogs of diacetyldianhydrogalactitol, dibromodulcitol, derivatives of dibromodulcitol, and analogs of dibromodulcitol. More typically, the DNA crosslinking hexitol is selected from the group consisting of dianhydrogalactitol, derivatives of dianhydrogalactitol, diacetyldianhydrogalactitol, derivatives of diacetyldianhydrogalactitol, dibromodulcitol, and derivatives of dibromodulcitol. In some embodiments, the DNA crosslinking hexitol is dianhydrogalactitol.

As used herein, the terms “substituted hexitol derivative” or “DNA crosslinking hexitol derivative,” “substituted hexitol,” “alkylating hexitol, “alkylating hexitol derivative,” and “DNA crosslinking hexitol” are used interchangeably herein and encompass these alternatives, unless specifically limited to a compound, a compound with defined substituents, or a class of compounds within the broad definitions provided above.

In some embodiments, the DNA crosslinking hexitol is dianhydrogalactitol, including its stereoisomers. The terms “dianhydrogalactitol,” “DAG,” and “VAL-083” are used herein interchangeably. The structure of dianhydrogalactitol (also referred to as DAG or VAL-083) is shown in Formula (I), below.

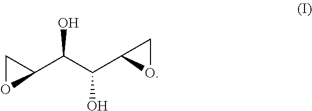

The galactitols, substituted galactitols, dulcitols, and substituted dulcitols included in the methods and combinations disclosed herein are either DNA crosslinking agents or prodrugs of DNA crosslinking agents, as discussed further below. Also within the scope of the invention are derivatives of dianhydrogalactitol that, for example, have one or both hydrogens of the two hydroxyl groups of dianhydrogalactitol replaced with lower alkyl, have one or more of the hydrogens attached to the two epoxide rings replaced with lower alkyl, or have the methyl groups present in dianhydrogalactitol and that are attached to the same carbons that bear the hydroxyl groups replaced with C2-C6 lower alkyl or substituted with, for example, halo groups by replacing a hydrogen of the methyl group with, for example a halo group. As used herein, the term “halo group,” without further limitation, refers to one of fluoro, chloro, bromo, or iodo. As used herein, the term “lower alkyl,” without further limitation, refers to C1-C6 groups and includes methyl. The term “lower alkyl” can be further limited, such as “C2-C6 lower alkyl,” which excludes methyl. The term “lower alkyl”, unless further limited, refers to both straight-chain and branched alkyl groups. These groups can, optionally, be further substituted, for example, with halo groups.

In some embodiments, the DNA crosslinking hexitol derivative is di acetyl dianhydrogalactitol. The structure of di acetyl dianhydrogalactitol is shown in Formula (II), below.

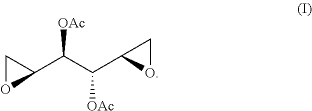 [SMILES: CC(=O)O[C@H]([C@H](OC(C)=O)[C@@H]1CO1)[C@@H]1CO1]

Also within the scope of the invention are derivatives of diacetyldianhydrogalactitol that, for example, have one or both of the methyl groups that are part of the acetyl moieties replaced with C2-C6 lower alkyl, have one or both of the hydrogens attached to the epoxide ring replaced with lower alkyl, or have the methyl groups attached to the same carbons that bear the acetyl groups replaced with lower alkyl or substituted with, for example, halo groups by replacing a hydrogen with, for example, a halo group.

In other embodiments, the DNA crosslinking hexitol derivative is dibromodulcitol of Formula (III):

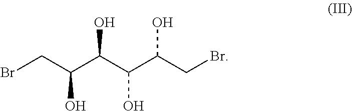 [SMILES: O[C@H]([C@H](O)[C@@H](O)CBr)[C@H](O)CBr]

Dibromodulcitol can be produced by the reaction of dulcitol with hydrobromic acid at elevated temperatures, followed by crystallization of the dibromodulcitol. Some of the properties of dibromodulcitol are described in N. E. Mischler et al., “Dibromoducitol,” Cancer Treat. Rev. 6: 191-204 (1979). In particular, dibromodulcitol, as a α,ω-dibrominated hexitol, dibromodulcitol shares many of the biochemical and biological properties of similar drugs such as dibromomannitol and mannitol myleran. Activation of dibromodulcitol to the diepoxide dianhydrogalactitol occurs in vivo, and dianhydrogalactitol may represent a major active form of the drug; this means that dibromogalactitol has many of the properties of a prodrug. Absorption of dibromodulcitol by the oral route is rapid and fairly complete.

Also within the scope of the invention are derivatives of dibromodulcitol that, for example, have one or more hydrogens of the hydroxyl groups replaced with lower alkyl, or have one or both of the bromo groups replaced with another halo group such as chloro, fluoro, or iodo.

The compounds described herein may contain one or more chiral centers and therefore, may exist as stereoisomers, such as enantiomers or diastereomers. The invention includes each of the isolated stereoisomeric forms (such as the enantiomerically pure isomers and other alternatives for stereoisomers) as well as mixtures of stereoisomers in varying degrees of chiral purity or percentage, including racemic mixtures and mixtures of diastereomers unless a specific stereoisomer is specified. Accordingly, the chemical structures depicted herein encompass all possible enantiomers and stereoisomers of the illustrated compounds including the stereoisomerically pure form (e.g., geometrically pure, enantiomerically pure or diastereomerically pure) and enantiomeric and stereoisomeric mixtures. When the chemical name does not specify the isomeric form of the compound, it denotes any one of the possible isomeric forms or mixtures of those isomeric forms of the compound.

Some compounds can also exist in several tautomeric forms, and the depiction herein of one tautomer is for convenience only, and is also understood to encompass other tautomers of the form shown. Accordingly, the chemical structures depicted herein encompass all possible tautomeric forms of the illustrated compounds.

As used herein, the term “solvate” means a compound formed by solvation (the combination of solvent molecules with molecules or ions of the solute), or an aggregate that consists of a solute ion or molecule, i.e., a compound of the invention, with one or more solvent molecules. When water is the solvent, the corresponding solvate is “hydrate.” Examples of hydrate include, but are not limited to, hemihydrate, monohydrate, dihydrate, trihydrate, hexahydrate, and other water-containing species. It should be understood by one of ordinary skill in the art that the pharmaceutically acceptable salt, and/or prodrug of the present compound may also exist in a solvate form. The solvate is typically formed via hydration which is either part of the preparation of the present compound or through natural absorption of moisture by the anhydrous compound of the present invention.

Additional derivatives of dianhydrogalactitol are known in the art. These derivatives include dimethyldianhydrogalactitol and disuccinyldianhydrogalactitol and are disclosed in Y. Zhou et al., “Research Progress in New Anti-Cancer Drugs with Hexitols,” Chin. J. Cancer 12: 257-260 (1993).

In some alternatives, the derivative or analog of dianhydrogalactitol can be a prodrug. As used herein, the term “prodrug” refers to compounds that are transformed in vivo to yield a disclosed compound or a pharmaceutically acceptable form of the compound. In some embodiments, a prodrug is a compound that may be converted under physiological conditions or by solvolysis to a biologically active compound as described herein. Thus, the term “prodrug” refers to a precursor of a biologically active compound that is pharmaceutically acceptable. A prodrug can be inactive when administered to a subject, but is then converted in vivo to an active compound, for example, by hydrolysis (e.g., hydrolysis in blood or a tissue). In certain cases, a prodrug has improved physical and/or delivery properties over a parent compound from which the prodrug has been derived. The term “prodrug” is also meant to include any covalently bonded carriers which release the active compound in vivo when the prodrug is administered to a subject. Prodrugs of a therapeutically active compound, as described herein, can be prepared by modifying one or more functional groups present in the therapeutically active compound in such a way that the modifications are cleaved, either in routine manipulation or in vivo, to yield the parent therapeutically active compound.

### Methods of Treatment and Compositions

As used herein, the terms “treat” and “treatment” refer to both therapeutic treatment and prophylactic or preventative measures, wherein the object is to prevent or slow down (lessen) an undesired physiological change or disorder, such as the growth, development or spread of cancer. For purposes of this invention, beneficial or desired clinical results include, but are not limited to, alleviation of symptoms, diminishment of extent of disease, stabilized (i.e., not worsening) state of disease, delay or slowing of disease progression, reducing disease recurrence, amelioration or palliation of the disease state, and remission (whether partial or total), whether detectable or undetectable. “Treatment” can also mean prolonging survival as compared to expected survival if not receiving treatment. Those in need of treatment include those already with the condition or disorder as well as those prone to have the condition or disorder or those in which the condition or disorder is to be prevented. The results of treatment can be determined by methods known in the art, such as determination of reduction of pain as measured by reduction of requirement for administration of opiates or other pain medication, determination of reduction of tumor burden, determination of restoration of function as determined by an improvement in the Karnofsky Performance Score, or other methods known in the art.

In some embodiments, the term “treatment” encompasses reduction in the invasiveness of the malignancy, including metastases.

As used herein, the term “synergistic” as used herein refers to a therapeutic combination which is more effective than the additive effects of the two or more single agents. A determination of a synergistic interaction between two or more agents, such as (i) dianhydrogalactitol, diacetyldianhydrogalactitol, or a derivative or analog thereof; and (ii) one or more additional chemotherapeutic agents, can be assessed using assays as known in the art. The combination therapy may provide “synergy” and prove “synergistic,” i.e., the effect achieved when the active ingredients used together is greater than the sum of the effects that results from using the compounds separately.

A synergistic effect may be attained when the active ingredients are: (1) co-formulated and administered or delivered simultaneously in a combined, unit dosage formulation; (2) delivered by alternation or in parallel as separate formulations; or (3) by some other regimen. When delivered in alternation therapy, a synergistic effect may be attained when the compounds are administered or delivered sequentially, e.g., by different injections in separate syringes or using other routes of administration. In general, during alternation therapy, an effective dosage of each active ingredient is administered sequentially, i.e., serially, whereas in combination therapy, effective dosages of two or more active ingredients are administered together. Combination effects can also be evaluated using both the BLISS independence model and the highest single agent (HSA) model (Lehar et al. 2007, Molecular Systems Biology 3:80). BLISS scores quantify degree of potentiation from single agents and a BLISS score >0 suggests greater than simple additivity. In some alternatives, combination effects can be evaluated using both the BLISS independence model and the highest single agent (HSA) model (Lehar et al. 2007, Molecular Systems Biology 3:80). BLISS scores quantify degree of potentiation from single agents and a BLISS score >0 suggests greater than simple additivity. An HSA score >0 suggests a combination effect greater than the maximum of the single agent responses at corresponding concentrations. An HSA score >0 suggests a combination effect greater than the maximum of the single agent responses at corresponding concentrations.

In one aspect, provided herein is a method for treating a malignancy, comprising (a) administering a therapeutically effective quantity of a DNA crosslinking hexitol and (b) administering a therapeutically effective quantity of a VEGF inhibitor to a patient with the malignancy to treat the malignancy. In certain embodiments, the administration of the therapeutically effective quantities of the hexitol derivative and the VEGF inhibitor reduces the invasiveness of the malignancy.

In another aspect, disclosed herein is a composition comprising (a) a DNA crosslinking hexitol and (b) a VEGF inhibitor.

In certain embodiments, the DNA crosslinking hexitol is dianhydrogalactitol.

The methods and compositions disclosed herein can be particularly used for treatment of gliomas, glioblastomas, and medulloblastomas.

The methods and compositions disclosed herein can be employed as either first-line or second-line therapy or can be used as adjunct therapy or in combination with another method of treatment and administered either concurrently or sequentially.

The amount of a given pharmacologically active agent, such as an DNA crosslinking hexitol derivative such as dianhydrogalactitol or an analog or derivative of dianhydrogalactitol as described above, and a VEGF inhibitor that is included in a unit dose of a pharmaceutical composition according to the present invention will vary depending upon factors such as the particular compound, disease condition and its severity, the identity (e.g., weight) of the subject in need of treatment, but can nevertheless be routinely determined by one skilled in the art.

As used herein, a “composition” can comprise one or more agents, such as dianhydrocalactitol and a VEGF inhibitor. The composition can comprise each of the agents combined in a single container with a pharmaceutically acceptable carrier, or the composition can comprise each of the active agents in a separate container with a pharmaceutically acceptable carrier, which can be either the same or different, wherein the composition comprises a treatment regimen.

In some embodiments, the therapeutically effective quantities are the quantities of the DNA crosslinking hexitol derivative and the additional agent, such as VEGF inhibitor, that produce synergism between the activities of the DNA crosslinking hexitol derivative and the additional agent.

When multiple therapeutic agents are administered according to the methods disclosed herein, each therapeutic agent can be administered separately, or two or more therapeutic agents can be administered in a single pharmaceutical composition. For example, when three therapeutic agents are to be administered, the following possibilities exist. (1) Each of the three therapeutic agents is administered individually; in this case, each agent can be administered in a separate pharmaceutical composition or as the agent alone without use of a pharmaceutical composition for the agent. (2) Two of the therapeutic agents are administered together in a single pharmaceutical composition, while the third therapeutic agent is administered separately, either as the agent alone or in a separate pharmaceutical composition. (3) All three therapeutic agents are administered together in a single pharmaceutical composition.

In some embodiments, administration of an effective dose of a DNA crosslinking hexitol can be done in any suitable manner, including continuous i.v. infusion for hours to days; weekly administrations; biweekly administration; doses greater than 5 mg/m²/day; progressive escalation of dosing from 1 mg/m²/day based on patient tolerance; doses less than 1 mg/m² for greater than 14 days; single and multiple doses escalating from 5 mg/m²/day via bolus; oral doses below 30 or above 130 mg/m²; oral or i.v. dosages up to 40 mg/m² for 3 days and then a nadir/recovery period of 18-21 days; dosing at a lower level for an extended period (e.g., 21 days); dosing at a higher level; dosing with a nadir/recovery period longer than 21 days; dosing at a level to achieve a concentration of the substituted hexitol derivative such as dianhydrogalactitol in the cerebrospinal fluid (CSF) of a clinically relevant concentration up to 5 μM; dosing at a level to achieve a cytotoxic concentration in the CSF for treatment of glioblastoma; administration on a 21-day to 33-day cycle with a cumulative dose of about 9 mg/m²; administration on a 21-day to 33-day cycle with a cumulative dose of about 10 mg/m²; administration on a 21-day to 33-day cycle with a cumulative dose of about 20 mg/m²; administration on a 21-day to 33-day cycle with a cumulative dose of about 40 mg/m²; administration on a 21-day to 33-day cycle with a cumulative dose of about 80 mg/m²; administration on a 21-day to 33-day cycle with a cumulative dose of about 160 mg/m²; administration on a 21-day to 33-day cycle with a cumulative dose of about 240 mg/m²; administration so that the plasma half-life is about 1-2 hours; administration so that the Cₘₐₓ is <200 ng/ml; and administration so that the substituted hexitol derivative has a half-life of >20 hours in the cerebrospinal fluid. When a 21-day to 33-day cycle is used in dosages as described above, typically a 21-day cycle is preferred.

In some embodiments, DNA crosslinking hexitol can administered at doses between about 20 mg/m² and about 40 mg/m² given IV on each of days 1, 2, 3 of a 21-day (3-week) cycle (resulting in a cumulative dose of between about 60 mg/m² and about 120 mg/m2 in the cycle). In certain embodiments, DNA crosslinking hexitol can be administered at 5 mg/m², 10 mg/m², 15 mg/m², 20 mg/m², 25 mg/m², 30 mg/m², 35 mg/m², or 40 mg/m² given IV on each of days 1, 2, 3 of a 21-day (3-week) cycle. The treatment cycle can be repeated as long as the patient continues to benefit from the treatment; for example, DNA crosslinking hexitol can be administered for 3 weeks, 6 weeks, 12 weeks, or 24 weeks as described above.

In certain embodiments, VEGF inhibitor treatment and DNA crosslinking hexitol treatment can be co-administered. In other embodiments, DNA crosslinking hexitol treatment can be administered following administration of one or more does of a VEGF inhibitor. In yet other embodiments, DNA crosslinking hexitol and VEGF inhibitors are administered on the same day or on different days. In some embodiments, when a malignancy has failed a VEGF inhibitor treatment, the methods disclosed herein comprise administering, as a third-line therapy after VEGF inhibitor treatment failure, a therapeutically effective quantity of a DNA crosslinking hexitol derivative, for example, DNA crosslinking hexitol selected from the group consisting of dianhydrogalactitol, diacetyldianhydrogalactitol, and dibromodulcitol. In these particular embodiments, administration of DNA crosslinking hexitol can be done as described above.

Ranges can be expressed herein as from “about” (or “approximate”) one particular value, and/or to “about” (or “approximate”) another particular value. As used herein, unless indicated otherwise, the term “about” refers to a range of values of plus or minus 5% of the value that follows the term. For example, the phrase “about 10” includes the range of values from 95% of 10 to 105% of 10, (from 9.5 to 10.5) unless clearly contradicted by context.

Typically, in the compositions and methods disclosed herein, the DNA crosslinking hexitol is dianhydrogalactitol (also referred herein as DAG or VAL-083).

The invention is illustrated by the following Examples. These Examples are included for illustrative purposes only, and are not intended to limit the invention.

### EXAMPLES

### Example 1

### Effect of Dianhydrogalactitol in Glioblastoma Cells Under Normoxic and Hypoxic Conditions

This Example is intended to show the effect of the administration of dianhydrogalactitol to glioblastoma cells under normoxic and hypoxic conditions.

The experiments described below were designed to answer whether dianhydrogalactitol increases its cytotoxic effects against glioblastoma cells under hypoxic conditions, such as by increasing DNA damage and cell cycle arrest, in vitro.

Without being bound by the hypothesis, if dianhydrogalactitol or a derivative or analog thereof is administered in combination with or immediately following bevacizumab or another VEGF inhibitor, the uptake of dianhydrogalactitol or the derivative or analog thereof can be increased due to the upregulation of one or more glucose transporters. This increased uptake would in turn increase the amount of drug getting into the tumor cells and thus the cell kill activity, without increasing the administered dose and systemic toxicity of dianhydrogalactitol. On the other hand, hypoxia has been shown to reduce sensitivity to other DNA-targeting and DNA crosslinking agents in GBM tumor cells. In addition, VEGF inhibitors reduce blood flow to the tumor and thus administration of dianhydrogalactitol in combination with or immediately following bevacizumab or another VEGF inhibitor it would be expected that dianhydrogalactitol's access to the tumor would be reduced. Thus, it was surprising that dianhydrogalactitol retained its activity in hypoxic cells. Moreover, dianhydrogalactitol-induced double-stranded DNA crosslinks that lead to cell death require cell cycling for the DNA double strands to form. Because hypoxic cells go through the cell cycle much slower (7-10 days) than normoxic cells (2-3 days), it was expected that dianhydrogalactitol would have lower cytotoxic efficacy under hypoxia in vitro. It was therefore surprising that the DNA damage, HR activation, and cell cycle arrest were equally high in hypoxic cells as in normoxic cells after 48-72 hours.

Dianhydrogalactitol readily crosses the blood-brain barrier and accumulates in brain tumor tissue and has demonstrated activity against glioblastoma in several historical clinical trials. In vitro, dianhydrogalactitol is active against glioblastoma cell lines with IC₅₀ values generally in low μM. Dianhydrogalactitol activity is independent of MGMT expression, it is equally active in glioblastoma cancer stem cells (CSC) and non-CSC, and it acts as a radiosensitizer in glioblastoma cancer cells and CSCs.

In vivo, dianhydrogalactitol increases median survival of mice with intracranial U251 xenografts. Dianhydrogalactitol is currently in clinical trial against recurrent glioblastoma and has demonstrated a clinically meaningful dose-response trend in glioblastoma patients who have failed both TMZ and bevacizumab. Additionally, dianhydrogalactitol has shown cell kill activity in glioblastoma cancer stem cells (CSCs) with IC₅₀ values within the expected concentration range in the CNS. CSC cells are extremely chemoresistant and are known to upregulate glucose transporters in response to hypoxia. A rationale for administration of dianhydrogalactitol or a derivative or analog thereof concurrent with or immediately following bevacizumab or another VEGF inhibitor, as this would increase the uptake of the dianhydrogalactitol or derivative or analog thereof in glioblastoma CSCs thereby increasing cell kill activity against CSCs and improving outcome of the disease.

Material and Methods

U251 cells were cultured as adherent monolayers in DMEM containing 10% FBS, 2 mM L-Glutamine and 100 U/ml Pen-Strep (all from Lonza). Classical normoxic cultures were performed at 37° C. under 5% CO2 atmospheric oxygen. Hypoxic conditions at 0.5% 02 were maintained in the hypoxic incubator chamber (Galaxy 48R incubator, New Brunswick, Canada). Hypoxic conditions were applied to cells 16 h before addition of dianhydrogalactitol and during 1 h treatment. Normoxic and hypoxic cells were treated with 50 uM dianhydrogalactitol for 1 h. After drug wash out all cultures were performed in normoxia and cells were collected at indicated time points after treatment.

For flow cytometry analysis, cells were dissociated and incubated with the IR-LIVE/DEAD® Fixable Dead Cell Stain (ThermoFisher scientific; 1 μg/ml) in HBSS, 2% FBS, 10 mM HEPES for 15 min. Cells were further fixed with BD Cytofix/Cytoperm™ (BD Bioscience) for 30 min and permeabilised with BD Perm/Wash Cytoperm™ (BD Bioscience) for 10 min. Cells were stained with conjugated antibodies: anti H2AX-Phospho Ser139-Alexa Fluor 647 (BD Biosciences, 560447) and anti ATMPhosphoSer1981-PE (Millipore, FCMAB110P). DNA was counterstained with DAPI (1 ug/ml). Data were acquired on a FACS Aria™ SORP cytometer (BD Biosciences) and analyzed with Diva (BD Biosciences) and FlowJo softwares.

For Western Blot analysis, cells were washed 2 times in cold HBSS buffer. Cells were collected in Eppendorffs and cell pellet was frozen in −80° C. for further study.

P3 and T16 GBM PDX-derived organotypic spheroids were recreated from 1000 single cells in a 384 well plate (SBio PrimeSurface, U-shape bottom, non-adherent). Cells were incubated overnight to reform 3D spheroids in DMEM medium, 10% FBS, 2 mM L-Glutamine, 0.4 mM NEAA and 100 U/ml Pen-Strep; all from Lonza. Classical normoxic cultures were performed at 37° C. under 5% CO₂ atmospheric oxygen in the Incucyte machine. Pictures were taken each hour. Spheroids were treated with a range of dianhydrogalactitol (lot 160801) concentrations for 72 h (1, 2, 4, 10, 30, 60, 100, 200, 300, 600, 1000μ M).

Cell viability in spheroids (n=3 per drug concentration) after 72 h of treatment was assessed by double labelling with 0.5μ M Calcein AM and 1μ M Ethidium homodimer-1 (LIVE/DEAD® Viability/Cytotoxicity assay kit, Molecular Probes™) for 4 hours. Measurements of viable (‘green’ florescence) and dead (‘red’ fluorescence) cells were performed using fluorescence confocal microscopy (Zeiss LSM STO META) by obtaining 20-25 stacks of two-dimensional images from successive focal planes (5 μm) for each spheroid.

Additionally, cell viability and cell death were assessed with the CellTiter-Glo 2.0 and CellTox Green Cytotoxicity assay (both from Promega), respectively (n=3 per drug concentration). The assays were performed according to manufacturer's protocols. Experiment was performed once with three technical replicates each.

Results

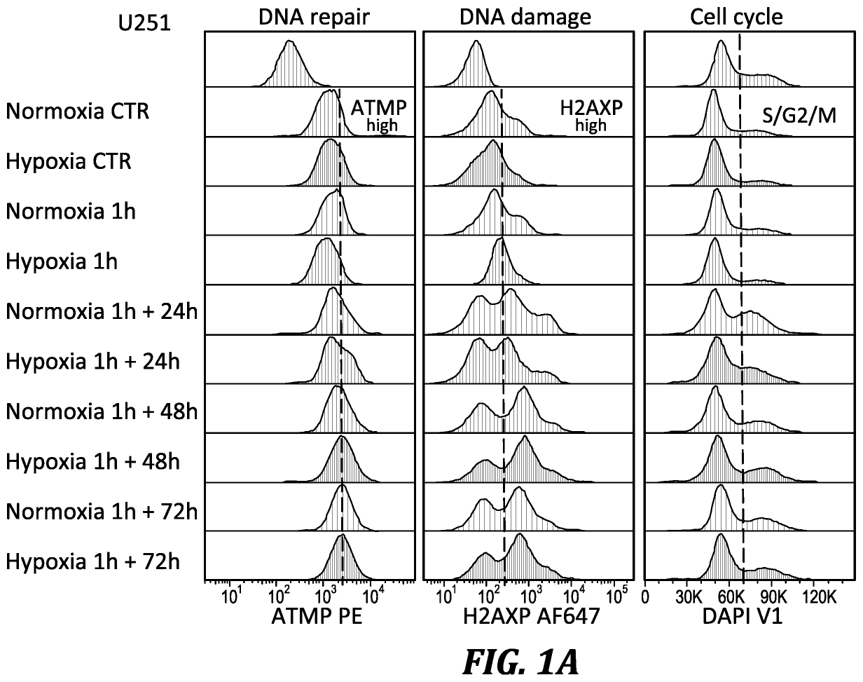

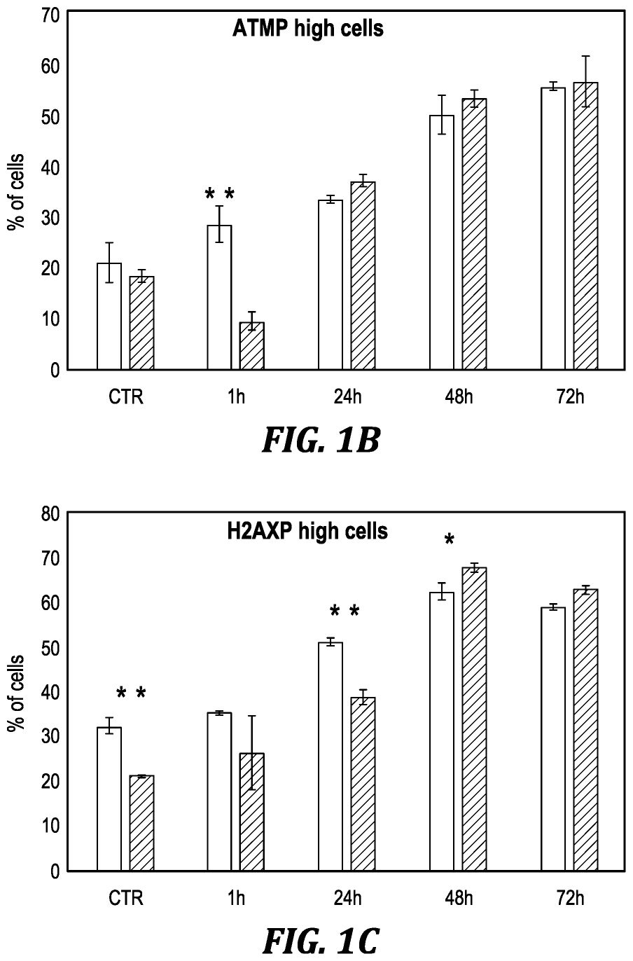

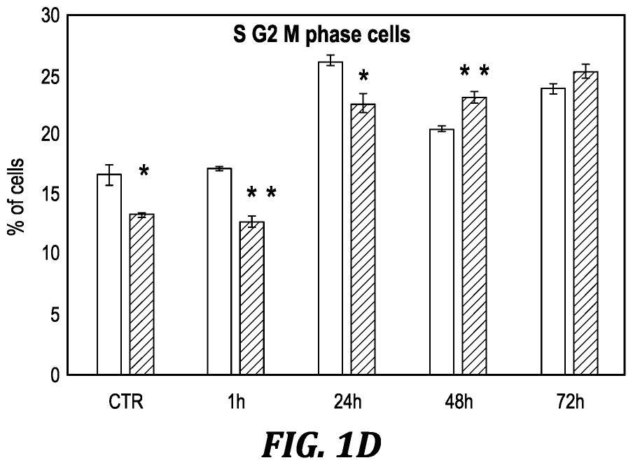

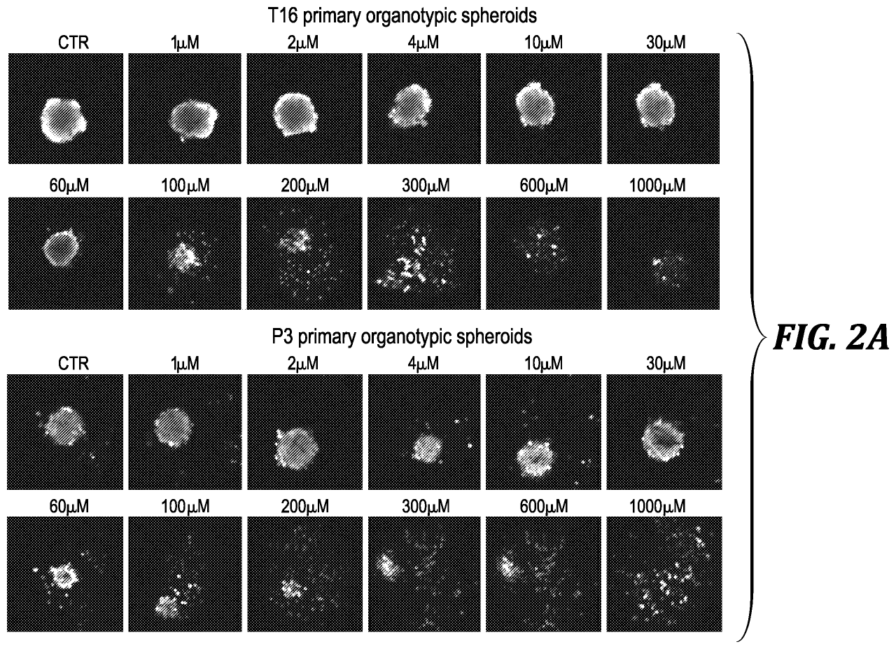

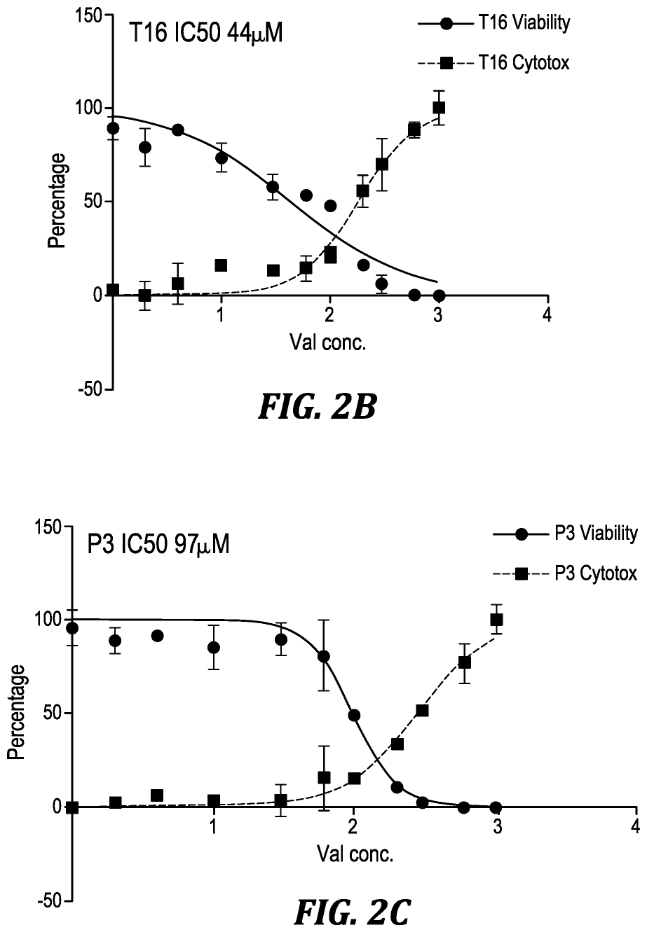

The results for the U251 studies are shown in FIGS. 1A-1D as a series of graphs showing the effect of dianhydrogalactitol on DNA repair, DNA damage, and cell cycling in hypoxic and normoxic cells of the U251 human glioblastoma cell line, with the data obtained by flow cytometry. In the graphs on the left side of FIG. 1A, ATMP denotes phosphorylated (=activated) ATM (ataxia-telangiectasia mutated) which is a kinase that is part of the homologous recombination (HR) DNA damage repair pathway; its presence is a marker for DNA repair, specifically through the HR pathway. PE is a fluorochrome that is used to detect the ATMP in flow cytometry. In the graphs in the middle panels of FIG. 1A, H2AXP denotes activated H2AX which is a histone marker for DNA double strand breaks (DNA damage). AF647 is a fluorochrome used for detection of activated H2AX in flow cytometry. In the graphs on the right side of FIG. 1A, DAPI is a fluorescent stain that binds to DNA and thus is a marker for the amount of DNA. It can therefore be used to detect cell cycle changes and the amount of DNA doubles through the S-phase and into the G2/M phase. Thus, in FIG. 1A, the leftmost panel shows the extent of DNA repair, the center panel shows the extent of DNA damage, specifically double-stand breaks, and the rightmost panel shows the proportion of cells at each point in the cell cycle. In FIG. 1A, the top panel shows the isotype antibody control. The following legends designating treatment of the cells are interpreted as follows: (i) “Normoxia CTR” is normoxic cells as a control without no dianhydrogalactitol treatment; (ii) “Hypoxia CTR” is hypoxic cells as a control without no dianhydrogalactitol treatment; (iii) “Normoxia 1 h” is normoxic cells after 1 hour of treatment with dianhydrogalactitol; (iv) “Hypoxia 1 h” is hypoxic cells after 1 hour of treatment with dianhydrogalactitol; (v) “Normoxia 1 h+24 h” is normoxic cells after a 1 hour pulse with dianhydrogalactitol followed by a 24-hour washout; (vi) “Hypoxia 1 h+24 h” is hypoxic cells after a 1-hour pulse with dianhydrogalactitol followed by a 24-hour washout; (vii) “Normoxia 1 h+48 h” is normoxic cells after a 1-hour pulse with dianhydrogalactitol followed by a 48-hour washout; and (vii) “Hypoxia 1 h+48 h” is hypoxic cells after a 1-hour pulse with dianhydrogalactitol followed by a 48-hour washout. In the graphs on the right side of FIG. 2, for each pair of bars, the left member of the pair is for normoxic cells, while the right member of the pair is for hypoxic cells. FIG. 1B shows the fraction of cells with high ATMP, i.e. high DNA repair activity, FIG. 2C shows the fraction of cells with high H2AXP, i.e., high number of DNA double strand breaks, and FIG. 1D shows the fraction of cells in the S/G2/M phases of the cell cycle (cells that are replicating or have replicated DNA).

The results of this example indicate that the cell cycle is decreased or paused in hypoxia. Treatment with dianhydrogalactitol leads to cell cycle arrest in S/G2/M but increased inhibition in hypoxic cells, which is observed only after 48 hours following cessation of administration of the dianhydrogalactitol. At 1 hour and 24 hours after dianhydrogalactitol treatment, cancer cells under normoxic conditions display more DNA damage (H2AXP) than hypoxic cells. However, after 48 (and possibly 72) hours, hypoxic cells have more damage than the normoxic cells. This supports the hypothesis that dianhydrogalactitol causes DNA double-strand breaks when the cell cycles through S-phase. Hypoxic cells are stalled in their cell cycle and thus do not acquire as much DNA damage after dianhydrogalactitol treatment as normally cycling cells under normoxic conditions would acquire. However, when the hypoxic cells, after 1 hr. dianhydrogalactitol treatment, are transferred to normoxic conditions, they slowly start cycling again and thus reach S-phase and accumulate DNA double strand breaks. Thus, hypoxic cells may take up more dianhydrogalactitol than normoxic cells but the resulting DNA damage does not become evident until the cells begin cycling, such at 48 to 72 hours.

The results for the T16 and P3 studies are shown in FIGS. 2A-2B. P3 and T16 organotypic spheroids were sensitive to dianhydrogalactitol at doses <100 μM. T16 spheroids were more sensitive to dianhydrogalactitol with IC₅₀=44 μM, compared to P3 (IC₅₀=97 μM). Cell death was observed already 4-6 h after treatment start for highest drug concentrations (not shown). FIG. 2A shows representative images of treated spheroids; ‘green’=viable cells, ‘red’=dead cells. FIGS. 2B and 2C show the cell viability and cytotoxicity in T16 and P3, respectively, upon exposure to dianhydrogalactitol. Due to lower IC₅₀, T16 model was chosen for the in vivo study (see below).

Summary

The aim of the study was to assess the cytotoxic activity of dianhydrogalactitol against GBM U251 cells under normoxia vs. hypoxia and against GBM PDX-derived spheroids P3 and T16 under normoxia. As expected, dianhydrogalactitol pulse-treatment for 1 hour increased formation of DNA double strand breaks, activation of the HR DNA damage repair pathway and cell cycle arrest in the S/G2 phase under normoxia and the effect was persistent up to 72 hours post-treatment. Under hypoxia, the effects of dianhydrogalactitol treatment were slower as expected due to slower cell proliferation. However, 48 and 72 hours post-treatment, the DNA damage, HR activation and number of cells in S/G2 arrest was equal to or surpassing those under normoxia, suggesting that dianhydrogalactitol retains its cytotoxic activity under hypoxia in spite of reduced cell proliferation and general chemoresistance in hypoxic GBM cells.

### Example 2

### Effect of Dianhydrogalactitol as Single Agent and in Combination with Bevacizumab in T16 PDX-Derived GBM Xenograft, In Vivo

Objective

The aim of the study was to assess the effect of dianhydrogalactitol combination treatment with bevacizumab in vivo in an orthotopic GBM PDX model. The response to dianhydrogalactitol treatment was tested alone and in combination with bevacizumab to determine whether bevacizumab-induced hypoxia in vivo increased dianhydrogalactitol uptake/efficacy.

Material and Methods

Serial transplantation of PDXs mice were used to expand the tumour material and prepare spheroids, as previously described (Bougnaud et al., 2016). T16 GBM spheroids were orthotopically implanted into the right frontal lobe of Swiss nude mice (6 per mice, total of 28 mice). Animals were monitored daily and the following criteria were evaluated: (1) loss of >10% of body weight, (2) exhibition of strong neurological signs (3) increased lordosis or (4) swollen belly. Tumor growth was monitored by MRI (T1- and T2-weighted MRI protocol; 3T MRI system, MR Solutions).

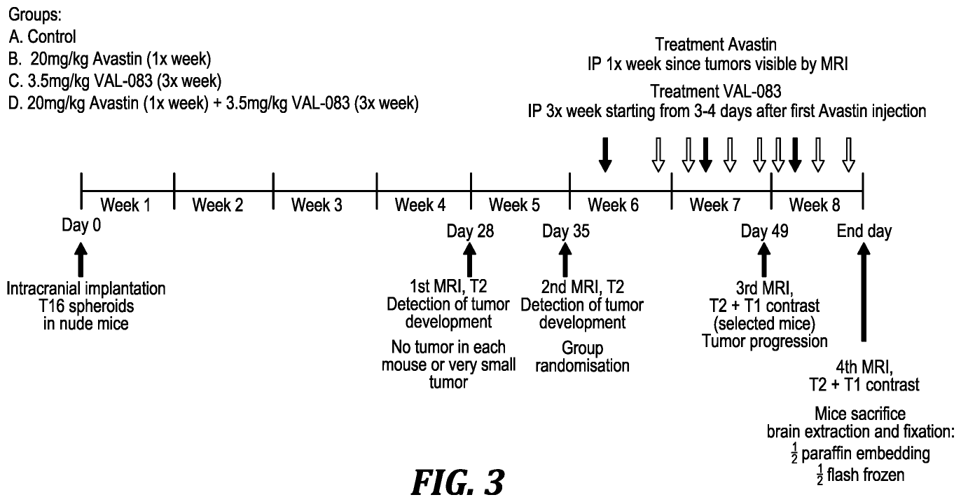

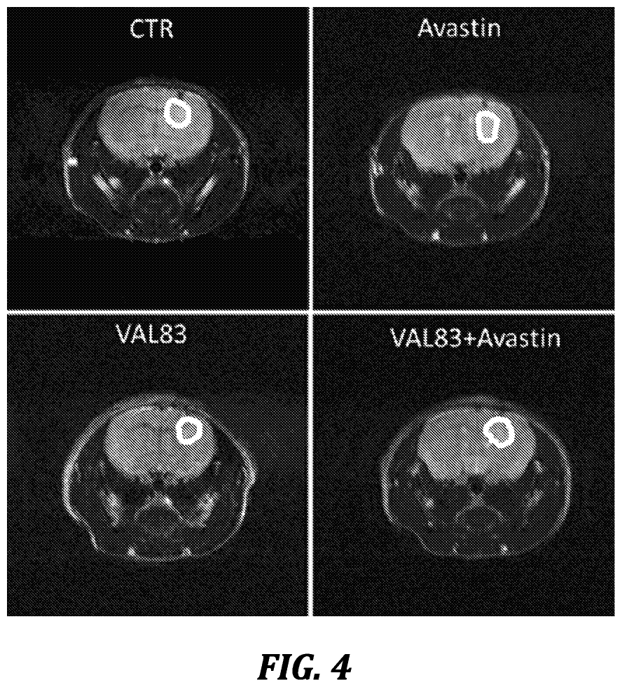

The final experimental schedule is shown in FIG. 3.

At day 28 post-implantation MRI did not show tumors yet in all mice, therefore randomization was not possible. One of the mice displayed possible ventricle injection of the tumor cells and was excluded from the study.

At 35 days post-implantation, most mice had visible tumors and mice were randomized into 4 treatment groups (7 mice per treatment group, 6 mice per control group): Control, bevacizumab treatment, dianhydrogalactitol treatment and combined bevacizumab+dianhydrogalactitol treatment.

Drug concentrations and treatment schedule were as follows:

1. Bevacizumab—20 mg/kg, 1×/week, first injection directly after randomization,

2. Dianhydrogalactitol—3.5 mg/kg, 3×/week, first injection 3 days after initial bevacizumab treatment, fresh compound each time

3. Normal saline (NaCl 0.9%)—4× week for all control group

Compounds and saline were delivered by intraperitoneal injections. Bevacizumab and dianhydrogalactitol injections were performed on different days.

49 days after implantation MRI (T2) was applied to monitor tumor progression. T1 with contrast agent was applied to several mice to evaluate the response of tumor to bevacizumab.

56 days after implantation one mouse in control group showed neurological symptoms and was euthanized directly after MM. T2 and T1+contrast MRI was applied to all mice.

Remaining mice were euthanized the following day and brains extracted. Control and bevacizumab-treated brains were stored: ½ brain for paraffin embedding, ½ flash frozen. Due to small tumor volume in dianhydrogalactitol treatment groups entire brains were extracted and stored for paraffin embedding (4 brains per group) or flash frozen (3 brains per group).

Tumor volume (mm³) was measured in ImageJ as the sum of area obtained by tumor delineation in each slice and multiplying by slice thickness (1 mm). Tumor growth rate (GR) was calculated using the TV measurement at the first and last time points as GR=100*log (TVf/TV0)/(tf−t0), where TVf and TV0 are the tumor volumes at the last and first time points respectively, and tf-t0 is the difference in days between the time points. Tumor volumes are expressed in mm3 and GR in ‘% per day’ (Obrad et al., JCBFM, 2017).

Results

T16 PDX model was chosen for the in vivo study. All mice carried tumors as detected by MRI at 35 days post-implantation. Mice were randomized into 4 treatment groups, 6 mice in each group, as shown in FIG. 3, and first bevacizumab treatment was applied directly to ensure induction of hypoxia in vivo before first dianhydrogalactitol injection. dianhydrogalactitol treatment started 3 days after first bevacizumab injection.

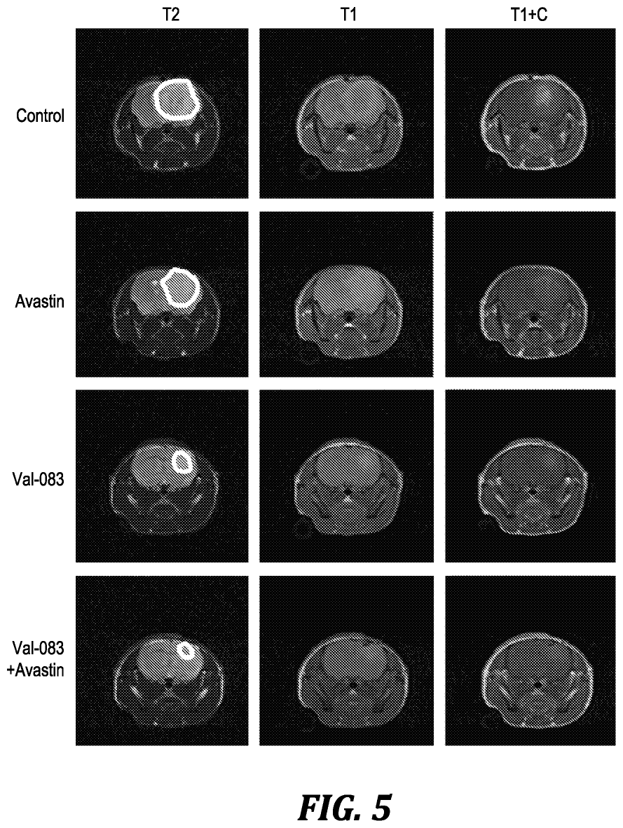

As expected, clear tumor progression was observed in Control and bevacizumab-treated mice, with difference towards smaller tumors upon bevacizumab treatment at the end of the study (−33%) (see FIGS. 5, 6 and 7). Bevacizumab treatment led to a clear decrease in contrast enhancement, confirming its efficacy in the experiment (FIG. 5). This is in accordance to our previous data showing vessel normalization leading to increased in vivo hypoxia upon bevacizumab treatment.

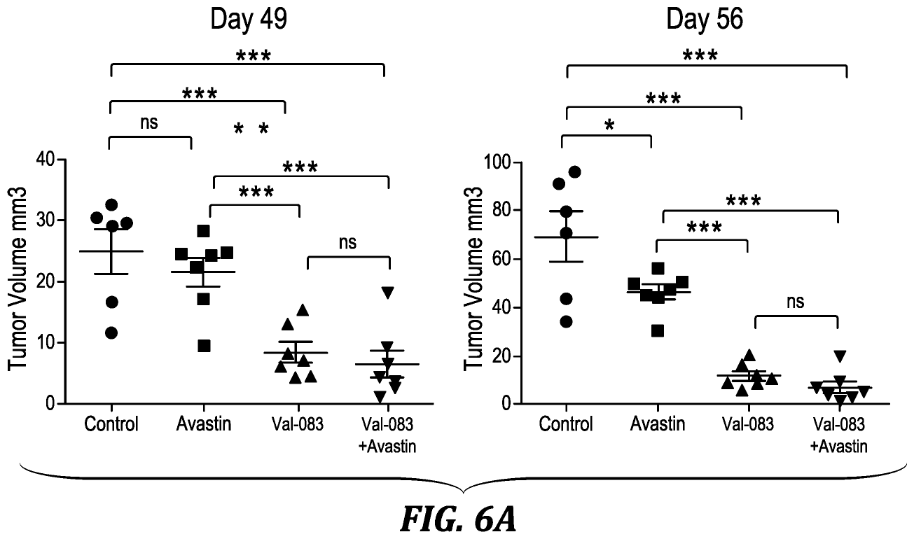

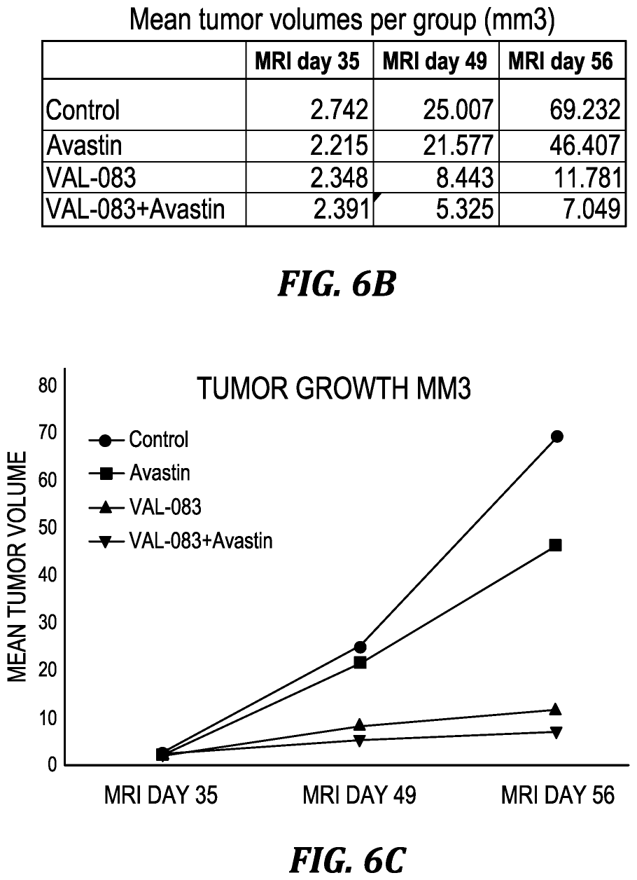

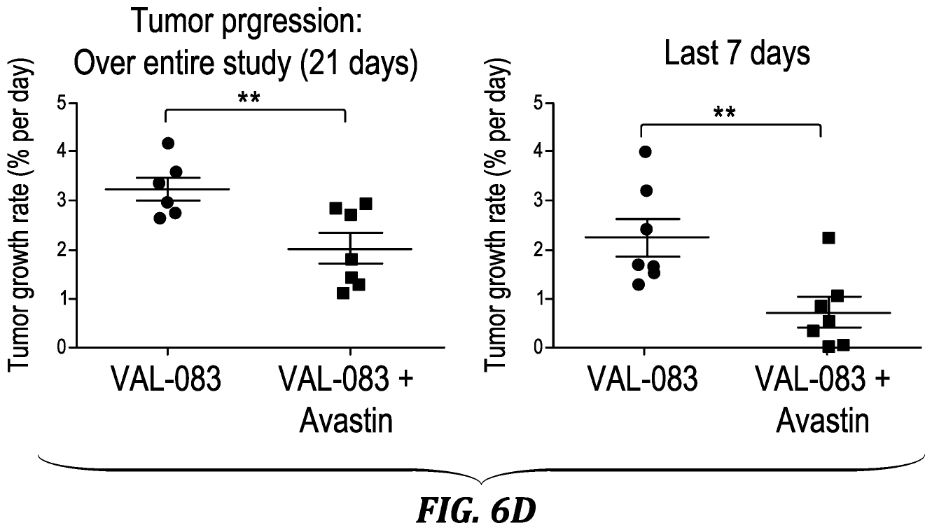

The in vivo study demonstrated that the combination was well tolerated by the animals. Dianhydrogalactitol treated groups showed significantly smaller tumors compared to Control and bevacizumab groups (−83% for dianhydrogalactitol group and −90% for dianhydrogalactitol+bevacizumab, FIGS. 6A and 6B). Thus, dianhydrogalactitol showed a strong effect on tumor progression as single agent and in combination with bevacizumab. Although no significant difference between tumor volumes was detected while comparing dianhydrogalactitol treatment and dianhydrogalactitol+bevacizumab groups at days of MRI (FIGS. 6A and 6B), the analysis of tumor growth in time showed significantly reduced tumor growth rate between the two groups (FIGS. 6C and 6D). These results indicate that treatment with VAL-083/bevacizumab combination may slow down tumor progression and an additive/synergistic effect with bevacizumab may be better visible at a lower dianhydrogalactitol concentration.

The results of Example 2 support the use of dianhydrogalactitol with a VEGF inhibitor, such as bevacizumab, which inhibits neovascularization of tumor cells and can result in cells assuming a hypoxic state.

Methods according to the present invention possess industrial applicability for the preparation of a medicament for the treatment of glioma and other malignancies, particularly malignancies of the central nervous system. Compositions according to the present invention possess industrial applicability as pharmaceutical compositions, particularly for the treatment of glioma and other malignancies, particularly malignancies of the central nervous system.

The method claims of the present invention provide specific method steps that are more than general applications of laws of nature and require that those practicing the method steps employ steps other than those conventionally known in the art, in addition to the specific applications of laws of nature recited or implied in the claims, and thus confine the scope of the claims to the specific applications recited therein. In some contexts, these claims are directed to new ways of using an existing drug.

The inventions illustratively described herein can suitably be practiced in the absence of any element or elements, limitation or limitations, not specifically disclosed herein. Thus, for example, the terms “comprising,” “including,” “containing,” etc. shall be read expansively and without limitation. Additionally, the terms and expressions employed herein have been used as terms of description and not of limitation, and there is no intention in the use of such terms and expressions of excluding any equivalents of the future shown and described or any portion thereof, and it is recognized that various modifications are possible within the scope of the invention claimed. Thus, it should be understood that although the present invention has been specifically disclosed by preferred embodiments and optional features, modification and variation of the inventions herein disclosed can be resorted by those skilled in the art, and that such modifications and variations are considered to be within the scope of the inventions disclosed herein. The inventions have been described broadly and generically herein. Each of the narrower species and subgeneric groupings falling within the scope of the generic disclosure also form part of these inventions. This includes the generic description of each invention with a proviso or negative limitation removing any subject matter from the genus, regardless of whether or not the excised materials specifically resided therein.

In addition, where features or aspects of an invention are described in terms of the Markush group, those schooled in the art will recognize that the invention is also thereby described in terms of any individual member or subgroup of members of the Markush group. It is also to be understood that the above description is intended to be illustrative and not restrictive. Many embodiments will be apparent to those of in the art upon reviewing the above description. The scope of the invention should therefore, be determined not with reference to the above description, but should instead be determined with reference to the appended claims, along with the full scope of equivalents to which such claims are entitled. The disclosures of all articles and references, including issued patents, published patent publications, and journal articles, are incorporated herein by this reference.

While illustrative embodiments have been illustrated and described, it will be appreciated that various changes can be made therein without departing from the spirit and scope of the invention.

The embodiments of the invention in which an exclusive property or privilege is claimed are defined as follows:

1. A method for treatment of a malignancy comprising administration of:
(a) a therapeutically effective quantity of an DNA crosslinking hexitol derivative selected from the group consisting of dianhydrogalactitol, diacetyldianhydrogalactitol, and dibromodulcitol; and
(b) a therapeutically effective quantity of a VEGF inhibitor.

2. The method of claim 1, wherein the administration of the hexitol derivative and the VEGF inhibitor reduces the invasiveness of the malignancy.

3. The method of claim 1, wherein the malignancy is a central nervous system malignancy.

4. The method of claim 3, wherein the central nervous system malignancy is a glioma, glioblastoma, or medulloblastoma.

5. The method of claim 1, wherein the DNA crosslinking hexitol derivative and the VEGF inhibitor are administered in any sequential order.

6. The method of claim 1, wherein the DNA crosslinking hexitol derivative and the VEGF inhibitor are administered on different days.

7. The method of claim 1, wherein the DNA crosslinking hexitol derivative and the VEGF inhibitor are administered on the same day.

8. The method of claim 1, wherein the VEGF inhibitor is bevacizumab.

9. The method of claim 1, wherein the DNA crosslinking hexitol is dianhydrogalactitol.

10. A method for treatment of a malignancy that has failed a VEGF inhibitor treatment comprising administering, as a third-line therapy after VEGF inhibitor treatment failure, a therapeutically effective quantity of a DNA crosslinking hexitol derivative selected from the group consisting of dianhydrogalactitol, diacetyldianhydrogalactitol, and dibromodulcitol.

11. The method of claim 1, wherein the malignancy is a glioma with p53 mutation, a glioma with an IDH wildtype, or a glioma with an IDH mutation.
